(19) United States
(12) Patent Application Publication
(10) Pub. No.: US 2008/0064999 A1
(43) Pub. Date: Mar. 13, 2008

(54) Wound Care Device

(76) Inventors: Kristian Larsen, Vaerloese (DK); Flemming Reissig Jensen, Vaerloese (DK)

Correspondence Address: JACOBSON HOLMAN PLLC, 400 SEVENTH STREET N.W., SUITE 600, WASHINGTON, DC, 20004, US

(21) Appl. No.: 11/632,651

(22) PCT Filed: Jul. 14, 2005
(86) PCT No.: PCT/DK05/00493

§ 371 Date: Jan. 16, 2007

(30) Foreign Application Priority Data
Jul. 16, 2004 (DK) PA 2004 01114

(51) Int. Cl. A61F 13/00

(52) U.S. Cl. 602/50; 602/48

(57) ABSTRACT

An absorbent wound care device comprising an absorbent element and an active agent, said absorbent element having a proximal and a distal surface, wherein the active agent is positioned on the proximal surface of the absorbent element and wherein the active agent is positioned in a zone having an exact defined area covering a central portion of the absorbent element, and wherein said area of the zone covers only a portion of the proximal surface of the absorbent layer, and a method of preparing such device.

### BACKGROUND OF THE INVENTION

1. Field of the Invention

The invention relates to a method of preparing a wound care device comprising an absorbent element and an active agent, and such wound care device, the wound care device being especially suitable for treatment of chronic wounds and burns.

Chronic wounds typically affect people over 65, due to the underlying clinical complications typically associated with chronic wounds, such as venous and arterial insufficiency, diabetes and trauma, and pressure sores caused by bed-rest. Besides the economic aspects of chronic wound treatment, the patients are suffering tremendously from a combination of infections, pain and low quality of life, combined with a significant increased risk of amputation of limbs and premature death.

Current treatment of chronic wounds and burns varies across the world, advanced wound healing devices based on passive bandages facilitating moist wound healing and exudate control being the current state of the art. It is therefore essential that efficient treatment of chronic wounds be developed, in particular treatments based on the active manipulations of problematic molecular factors in the wound contributing to chronicity through actively retarding the healing process.

2. Description of the Related Art

The majority of active medical devices used in wound care today involve incorporation of active agents as an integrated part of the entire product. This may lead to unnecessary breakdown of the active agent due to harsh chemical and physical conditions often encountered during the manufacture of the device. It is also known that accurate dosing of the active agent can be a problem for such devices, this is especially pronounced when working with low dose products, e.g. when using highly potent agents where only small amounts are necessary for achieving the desired effect.

Also when the product is in use the waste of active agent is pronounced as the active agent is only released at the site of contact with wound fluid and not at the site of contact with intact skin. This also leads to an unnecessary-contact between the active agent and the intact skin resulting in a possible reaction/breakdown of the intact skin.

Furthermore, if the active agent is distributed in a thick dressing, a part of the active agent may be trapped in the dressing or never wetted and thus not released.

Compounds for enhancing wound healing, such as hormones, proteins and peptides, may often be fragile and sensitive to many of the processes during the preparation of wound care devices.

Many attempts have been made in order to incorporate sensitive substances into wound care devices, such as absorbent foams. One method is to impregnate a foam by dipping the foam into a solution of the active agent, and then subsequently drying and evaporating the solvent. The drying process however, often requires exposure to elevated temperatures for prolonged periods of time and may be detrimental to the fragile active agent.

Another way of incorporation is to mix the active agent with the reactants for the foam material in situ, i.e. the active agent is present during the foaming reaction. These conditions expose the active agent to a very reactive chemical environment in which the active agent may be susceptible to degradation, denaturation and/or chemical modification. Also, considering the harsh conditions applied in the subsequent drying process, loss of activity is most likely and represents a major concern employing this process.

If the active agent is incorporated in the adhesive it may be covering the whole dressing as seen in dressings known in the art, and not being located in a distinct area, such as covering the wound but not the surrounding healthy skin. Thus the healthy skin may be exposed to the active agent, which is undesirable.

The active agent may be incorporated in a contact layer overlying the absorbent layer. Such layer will, however, typically be a part of the dressing and cover the entire skin-facing surface of the dressing, as it may be technically difficult to apply it only to the wound bed area.

Compounds such as those mentioned above are expensive, and even though only small amounts sometimes may be needed, cost is an important consideration.

It would be desirable to develop devices and methods for reliably delivering suitable active agents, therapeutic agents or bioactive materials directed and delivered only towards the local area of treatment with a minimum of waste of active agent and undesired skin or tissue exposure.

Applying an amount (often a small amount) of active agent onto a restricted area of an absorbent device may be difficult. In British patent applications Nos. GB 2 268 907 and GB 2 251 209 a method of printing on foam is disclosed. The print is deposited on a carrier, and then transferred from the carrier to the foam by the application of heat and pressure. The method is laborious and not suitable for sensitive compositions, as they may suffer from the heat and pressure treatment.

Thus there is still a need for a method of preparing a wound care device with an active agent incorporated locally over the wound.

### SUMMARY OF THE INVENTION

The object of the present invention is to facilitate accurate dosing of an active agent (or a chemical entity) on to an absorbing element capable of releasing the agent to a wound.

Another object of the present invention is to apply an active agent (or a chemical entity) on a well-defined area of an absorbing element.

Another object is to prevent undesired exposure of skin and tissue to the active agent.

Another object of the invention is to minimize waste of active agent (or a chemical entity) during production.

Yet another object of the invention is to protect an active agent (or a chemical entity) from harsh chemical conditions resulting in breakdown and loss of activity during production of the wound care device.

Still another object of the invention is to provide controlled release of an active agent (or a chemical entity) from the absorbing element.

### BRIEF DESCRIPTION OF THE DRAWINGS

The invention is disclosed more in detail with reference to the drawings in which

FIG. 1a shows an embodiment of the invention seen from above,

FIG. 1b the same embodiment in cross-section,

FIG. 2 shows another embodiment of the invention seen from above, and

FIG. 3 shows a third embodiment of the invention seen from above.

### DETAILED DESCRIPTION OF THE PRESENT INVENTION

The invention relates to a method of preparing an absorbent wound care device, said method comprises the steps of:

providing a wound care device comprising an absorbent element, said element having a proximal and a distal surface,

providing an active agent,

applying the active agent by spray coating to a central portion of the proximal surface of the absorbent element in a zone having an exact defined area.

By applying the active agent by spray-coating the agent may be applied to the surface of the absorbent element in an exact amount and a definite area, without waste of the agent. Furthermore, the agent may be located on the surface and/or the upper part of the absorbent element, decreasing the risk of waste of agent by entrapment in the deeper parts of the absorbent layer.

By spray dosing the agent may be applied to a definite area of the absorbent layer, by knowing the extent of the spray zone or by restricting the spray by using a stencil. The stencil may be provided with a pattern so the agent will be coated on the layer in this pattern.

In a preferred embodiment of the invention the active agent is applied by the use of Low Volume Low Pressure (LVLP) technology. LVLP coating comprises a precision spray valve, a microprocessor-based valve controller and a fluid reservoir. They operate on compressed air or nitrogen, and electricity. Either a 5-24V DC signal or a mechanical contact switch may initiate the spray cycle.

The spray valve is an air actuated needle type valve with two air inputs, one to actuate the valve, and the other to control fluid distribution. The LVLP design uses a low fluid flow rate so that only very low air pressure is required to apply the fluid as a soft spray. This approach provides high transfer efficiency without over-spray or mist. Changing the nozzle and air cap may produce a variety of spray patterns.

During system operation, actuating air from valve controller acts on a piston that retracts the needle from the nozzle seat, allowing fluid to flow from the nozzle. At the same time, low-pressure nozzle air (also from the controller, and typically in the 1-3 psi range) is turned on and flows from an annulus around the nozzle. This creates a pressure drop around the nozzle, causing fluid to break into fine droplets. The valve controller makes it simple to adjust nozzle air pressure for different coating materials and viscosities.

When the actuating air is shut off, the needle moves into the nozzle seat and shuts off the flow of fluid. Nozzle air continues for a split second to provide a clean cut off and ensure clog-free operation.

The LVLP technique provides consistent coating of low to medium viscosity fluids and microlitre to millilitre amounts can be reliably dispensed in round patterns with diameters ranging from 4 to 80 mm and in fan patterns with widths up to 165 mm. The spray-coating technology is capable of handling liquids and dispersions of a wide range of viscosities

The main part of the active agent of the zone is deposited on the surface of the absorbent element. However, depending on the structure of the absorber, the amount and the viscosity of the spraying liquid, some of the agent may penetrate below the surface of the absorbent element and be located in the upper part of the absorbent layer. Preferably 90% of the agent may be located in the upper 25% portion of the absorbent element, more preferably 90% may be located in the upper 10% portion of the absorbent element, even more preferably 90% may be located in the upper 5% portion of the absorbent element and most preferred 90% may be located in the upper 2% portion of the absorbent element.

The wound care device may be sterilized before applying the active agent. It is often desired to sterilize wound care devices, such as wound dressings. However, when incorporating components being sensitive to heat and/or radiation in the dressing, it may be difficult to sterilize the dressing without damaging the agent. Many peptides and proteins, e.g. enzymes, may be sensitive to sterilizations processes. By the method of the present invention it may be possible to prepare the wound care device, sterilize it and then apply the active agent aseptically. In the case that the active agent is insensitive to the sterilization processes it is of course possible to sterilize after application of the agent.

The active agent may be homogenously distributed within the zone, or it may be applied in a predetermined pattern. The concentration of the-active agent may be the same all over the zone, or the agent may be applied in a gradient, e.g. with a higher concentration at the central portion, encircled by one or more zones of lower concentration.

By coating the wound care device with a thin layer of a solution or dispersion of active agent only a low amount of agent is necessary as the waste is negligible. The amount of solvent used for the spray coating is so low that heating in order to evaporate the solvent may be avoided. Furthermore, the active agent may be applied in an aseptic manner, thus avoiding the need for sterilization.

The dosage of the agent is easy and accurate even at very low doses, and waste of the (often very expensive) agent is minimized.

Only the wound is treated, not the surrounding skin due to the limited and precise location of the agent on the wound care device.

In one embodiment of the invention a gel-forming compound, such as a polymer, may also be applied to the device. The gel-forming polymer may serve as a vehicle for the active agent, and may, although not to be considered as limiting for the invention, comprise of a solution of a gel-forming polymer, such as hyaluronic acid, chitosans, PVP or Na-CMC.

The gel-forming polymer may both serve as a moisturizer of the wound bed and as diffusion controllable layer of the active agent, thereby providing controlled release. Furthermore, the gel-forming polymer may provide a non-stick wound contacting surface of the device.

Preferably, the gel-forming polymer is applied within the zone of active agent. However, it may be distributed in a zone extending further than the active agent or it may cover a smaller area than the active agent.

In an embodiment of the invention the gel-forming polymer is applied after the active agent. In this way the polymer may serve as a wound-contacting layer and/or provide a controlled release of the active agent.

The gel-forming polymer may also enhance the hydrophilicity of the device, thereby providing the device with an increased initial absorption. This is often desired when handling exuding wounds. Furthermore, the polymer provides a soft and skin-friendly surface against the wound.

In one embodiment of the invention the gel-forming polymer is provided together with the active agent, e.g. in a solution or dispersion to be sprayed to the device.

In order to be able to spray-coat the active agent to the device, the agent may be provided in the form of a dispersion or solution. The solvent or dispersion medium may be chosen with regard to the properties of the active agent. After application, the solvent/dispersion medium may be removed, e.g. by evaporation. As the method of the invention renders it possible to apply small amounts of agent in a thin layer, as well as a major part of the solvent may evaporate during the spray-coating process, the amount of residual solvent/dispersion medium on the device may be almost negligible, and severe heat treatment or the like in order to remove the solvent may be avoided.

The invention also relates to a wound care device comprising an absorbent element and an active agent, said absorbent element having a proximal and a distal surface, wherein the active agent is positioned on the proximal surface of the absorbent element and wherein the active agent is positioned in a zone having an exact defined area covering a central portion of the absorbent element, and wherein said area of the zone covers only a portion of the proximal surface of the absorbent layer.

It has surprisingly been shown that by combining the active agent in a limited zone with having the agent in a thin layer, being proximate to the wound, an effective utilization of the active agent is achieved. Maximal utilization of active agent by targeted delivery of the agent to the wound is achieved.

By having the active agent in such zone it is possible to control the dose of agent to which the wound is exposed to, as the major part of the agent will be close to or in direct contact with the wound. Furthermore it is easier to dose the agent to the dressing.

The present invention recognizes and addresses some of the foregoing drawbacks, and deficiencies of prior art constructions and methods.

The invention also relates to the manufacture of a compound for treatment of chronic wounds or burns.

The invention further relates to a controlled delivery system of an active agent. This active agent may, although not to be considered as limiting for the invention, be selected from the group of compounds capable of inhibiting human neutrophil elastase.

It is preferred that the active agent is distributed on the surface of the absorbent element in a rather thin layer. This renders it possible to minimize the amount of active agent used, as almost all of the agent will be in the contact area and not deeply embedded in the dressing. In one embodiment of the invention the ratio between the area of the active agent zone and the thickness of the zone is at least 7000, preferably 10000, more preferably 20000.

The active agent may be distributed on the surface in different patterns. In one embodiment of the invention the active agent may be homogenously distributed within the zone. In another embodiment of the invention the active agent may be positioned in a predetermined pattern. The pattern may e.g. be in the form of dots or lines, geometric or random patterns.

The absorbent layer may be prepared from any absorbent material being suitable for use in a wound care device. Preferably the absorbent element is selected from the group of foam, alginates, polysaccharides, chitosans, super absorbent materials or combinations thereof.

It may be desirable to provide the absorbent layer with a cover layer facing the wound site. This layer may serve as a wound-contacting layer, providing a non-stick surface to the wound or it may prevent the components of the absorbent layer from disintegrating into the wound. The active agent zone may be provided on the cover layer. The layer may also serve as a filter preventing slough from blocking the absorbent layer. The layer may be in the form of a net or web or permeable film, non-woven nor woven. The layer is preferably substantially non-absorbent, but in some cases it may be desirable that the cover layer may be absorbent, e.g. a hydrogel or a layer being soluble in the wound exudates. In one embodiment of the invention the device comprises a net-like layer.

The wound care device may further comprise a backing layer. The backing layer may preferably be water impervious but vapor permeable.

In one embodiment of the invention the dressing comprises a gel.

The active agent may be any suitable pharmaceutically or biologically active agent, such as antibacterial, antiseptic, proteins, enzymes, enzyme inhibitors, odor controlling, pain relieving etc or combinations thereof.

In one embodiment of the invention the active agent is selected from the group of enzymes, proteins, peptides and the like.

In a preferred embodiment of the present invention, the active agent is EPI-HNE1-4, 56 amino acid proteins (6231 Da), discovered using a technology called “Directed evolution of novel binding proteins”, is derived from the second Kunitz type domain of the light chain of the human inter-alpha-inhibitor protein (ITI-D2) [U.S. Pat. No. 5,663,143, and references herein, hereafter incorporated in its entirety as reference].

EPI-HNE1-4 is member of a family of potent elastase inhibitors. EPI-HNE1-4 has been modified in the N-terminal residue to facilitate secretion from the yeast species P. pastoris, in which it can be produced by fermentation.

In a preferred embodiment of the present invention, the active agent is Alpha-1-Antitrypsin (AAT).

The device of the present invention is especially suitable for active agents being unstable, fragile, sensitive and/or expensive. The active agent is provided in a zone being close to or in direct contact with the wound, and thus very little amount of active agent is wasted, compared to wound dressings wherein the active agent is distributed in the entire absorbent layer.

Furthermore, if the active agent is sensitive to e.g. sterilization by heat or radiation, it may be possible to apply the active agent layer to the device after the device has been sterilized.

The active agent may be present in an amount large enough to achieve an effect in the wound site. Depending on the choice of active agent very low doses of active agent may be enough.

The invention further relates to a method of treatment of a chronic wound or burns, by providing a wound care device comprising an absorbent element and an active agent, said absorbent element having a proximal and a distal surface, wherein the active agent is positioned on the proximal surface of the absorbent element and wherein the active agent is positioned in a zone having an exact defined area covering a central portion of the absorbent element, and wherein said area of the zone covers only a portion of the proximal surface of the absorbent layer and applying said device to the wound.

It has surprisingly been shown that by spray coating of an absorbent material a very narrow dose variation of the active agent can be obtained even at very low doses. Thus, a precise dosage may be obtained. Degradation problems of the active agent, often encountered during production of the absorbent material can be avoided, as the active agent will be coated on to the absorbent material postproduction.

By spray coating the active agent to the absorbent layer it is possible to control the spreading of the active agent. The active agent zone may be well defined and the agent is homogeneously coated to the device. Furthermore it renders it possible to apply small amounts of active agent in a controlled manner.

### DESCRIPTION OF THE PREFERRED EMBODIMENTS

The invention is now explained more in detail with reference to the drawings showing preferred embodiments of the invention.

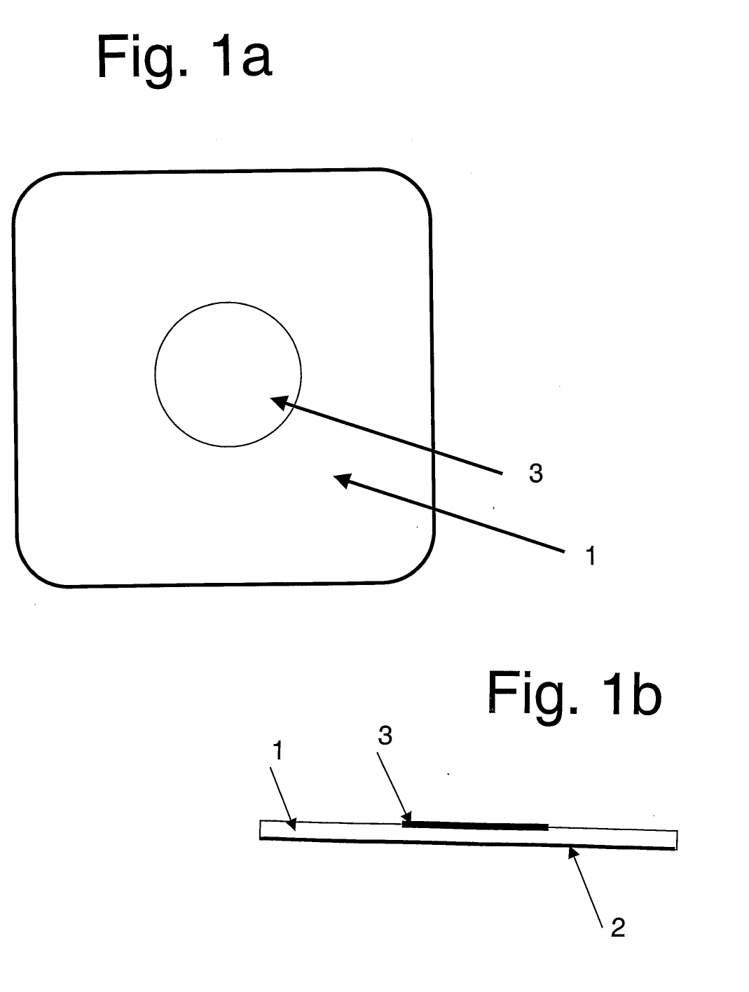

With reference to FIG. 1a and 1b, the device of the present invention may be a wound dressing comprising an absorbent element (1), covered at the distal surface (with regard to a wound) a top layer (2), such as a water impervious moisture permeable film. The active agent is coated in a well-defined zone (3) at the central portion of the wound-facing surface of the absorbent element. The area of the zone is preferably approximately the size of the wound. The zone may comprise multiple layers (not shown) e.g. comprising a mixture of the active agent and a gel-forming polymer. The gel-forming polymer provides a controlled/retarded/slow release of the active agent when the device is applied to a wound site. The amount and concentration of the gel-forming polymeric substance is chosen so as to provide such control.

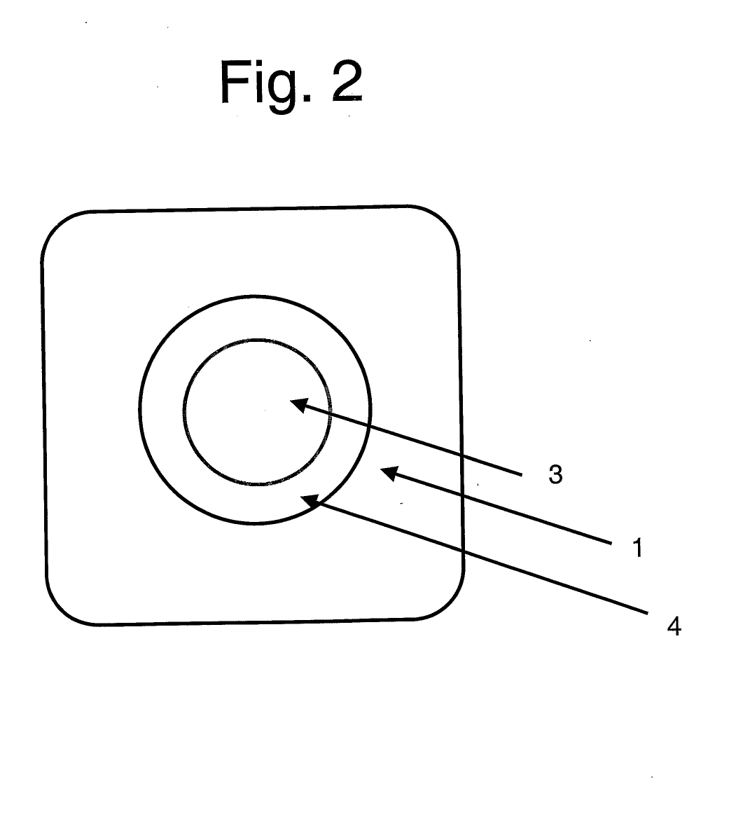

FIG. 2 shows a device of the present invention in the form of a wound dressing comprising an absorbent element (1). First a layer of active agent is applied in-a well-defined zone (3) and then a second zone (4), overlying and extending further than the first zone (3) is applied. The second zone (4) may be larger than the first zone (3) or vise versa. This second zone (4) may comprise a gel-forming composition, such as one or more gel-forming polymers. Apart from providing a controlled release of the active agent, the gel-forming polymer may also protect the patient form unnecessary exposure to the active agent on the intact skin, as the gel-forming polymeric layer is only degraded upon contact with wound fluid.

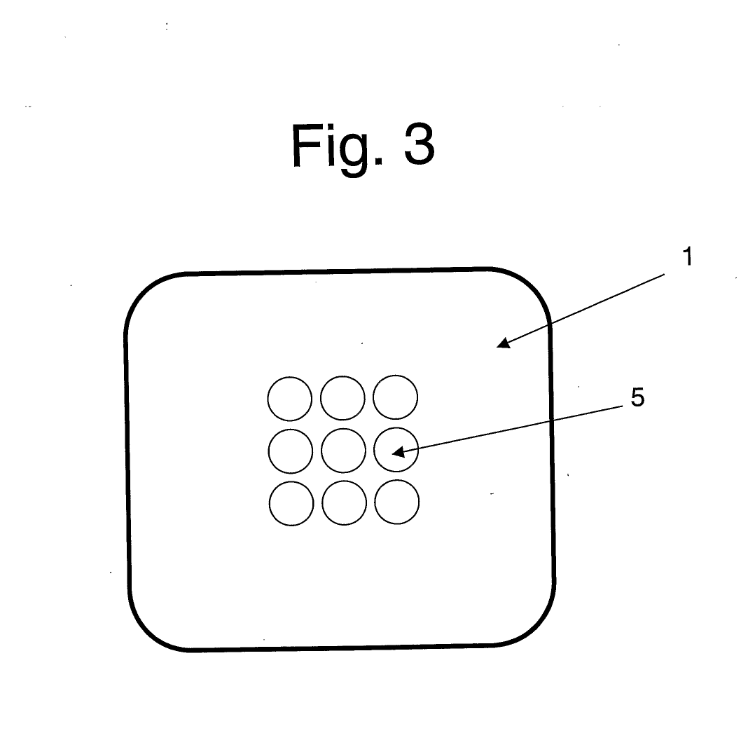

With reference to FIG. 3, the dressing comprises a zone of active agent in the form of a predetermined pattern, here shown as multiple discrete dots (5) having the active agent homogeneously distributed within each said dot. Such pattern may be achieved by use of an accurate spray nozzle or by using a stencil.

1. A method of preparing an absorbent wound care device, said method comprises the steps of:
providing a wound care device comprising an absorbent element, said element having a proximal and a distal surface,
providing an active agent,
applying the active agent by spray coating to a central portion of the proximal surface of the absorbent element in a zone having an exact defined area.

2. A method according to claim 1, wherein the wound care device is sterilized before applying the active agent.

3. A method according to claim 1, wherein the active agent is homogenously distributed within the zone.

4. A method according to claim 1, wherein the active agent is applied in a predetermined pattern.

5. A method according to claim 1, wherein a gel-forming polymer is applied after the active agent.

6. A method according to claim 1 wherein a gel-forming polymer is provided together with the active agent.

7. A method according to claim 1 wherein the active agent is provided in the form of a solution.

8. A method according to claim 1 wherein the active agent is provided in the form of a dispersion.

9. An absorbent wound care device comprising an absorbent element and an active agent, said absorbent element having a proximal and a distal surface, wherein the active agent is positioned on the proximal surface of the absorbent element and wherein the active agent is positioned in a zone having an exact defined area covering a central portion of the absorbent element, and wherein said area of the zone covers only a portion of the proximal surface of the absorbent layer.

10. A wound care device according to claim 11, wherein the ratio between the area of the zone and the thickness of the zone is at least 20000.

11. A wound care device according to claim 1, wherein the absorbent element is selected from the group of foam, alginates and super absorbent materials or combinations thereof.

12. A wound care device according to claim 1, wherein the device further comprises a net-like layer.

13. A wound care device according to claim 1, wherein the active agent is selected from the group of enzymes or proteins

14. A wound care device according to claim 1, wherein the device further comprises a gel-forming polymer.
